(19) United States
(12) Patent Application Publication
(10) Pub. No.: US 2024/0038338 A1
(43) Pub. Date: Feb. 1, 2024

(54) SYSTEM AND METHOD FOR AUTOMATED FLOW CYTOMETRY DATA ANALYSIS AND INTERPRETATION

(71) Applicant: Kamraan Gill, Norwalk, CT (US)

(72) Inventor: Kamraan Gill, Norwalk, CT (US)

(21) Appl. No.: 17/875,319

(22) Filed: Jul. 27, 2022

(51) Int. Cl. G16B 40/10; G16B 50/30

(52) U.S. Cl. CPC G16B 40/10; G16B 50/30

(57) ABSTRACT

An apparatus for analyzing flow cytometry data for a fluid sample has a datastore that stores cytometry datasets, a computing system coupled to the datastore, marker pairs, a user interface coupled with the computing system, a data structure representing a bivariate coordinate system having an x-axis, a y-axis, and an area, and, event populations (an initial population and one or more subpopulations). Each cytometry dataset has a series of events about the fluid sample. Each event has parameter values associated with one of a scatter parameter and an antigen parameter. The computing system operates upon the cytometry datasets. Each marker pair has a first and a second parameter. The area has units (cluster and empty units).

### CROSS REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

Flow cytometry provides a well-established method to identify cells in solution and may be used for evaluating peripheral blood, bone marrow, and other body fluids. Flow cytometry studies are used to identify and quantify immune cells and characterize hematological malignancies. In general, flow cytometry is a technique used to detect and measure physical and chemical characteristics of a population of cells or particles in a fluid sample. In this process, a sample containing cells or particles is suspended in a fluid and injected into the flow cytometer instrument. Flow cytometry may be used to diagnose blood cancers because cancer cells may gain or lose particular antigens. Identifying antigens that are present or absent from a cell allows abnormal (e.g., cancer) cells and normal (e.g., healthy) cells to be differentiated based on the presence or lack of certain antigen molecules.

In flow cytometry a set of antibodies may be selected based on each antibody's ability to bind to a specific antigen on a cell. The set of antibodies is added to a fluid sample to be diagnosed. In the fluid sample the antibodies attach to specific antigens if the antigens are present. The antibodies are tagged with fluorochromes (e.g., fluorescent molecules) that fluoresce at a specific wavelength. Detection of the fluorescence value corresponding to a specific wavelength indicates the presence of the antibody in a fluid sample and indicates the presence of a specific antigen. The fluid sample is run through a flow cytometer where the cells with any antibodies attached to each cell, flow single file through a fluorescence detector. The flow cytometer detects and records all fluorescence values for each cell of the fluid sample into a fluid sample cytometry dataset.

After the sample has finished running through the flow cytometer, the data is reviewed and analyzed by technologists, pathologists, and other medical personnel. A pathologist will often write a diagnostic report based on visual analysis of the flow cytometry fluid sample data.

The present invention provides systems and methods to automate the review and analysis of flow cytometry data and diagnostic report generation.

### SUMMARY OF THE INVENTION

In an embodiment of the present invention, an apparatus for analyzing flow cytometry data for a fluid sample comprises a datastore that stores one or more cytometry datasets, a computing system coupled to the datastore, one or more marker pairs, a user interface coupled with the computing system, a data structure representing a bivariate coordinate system having an x-axis, a y-axis, and an area, and, one or more event populations selected from a group consisting of an initial population and one or more subpopulations. Each cytometry dataset comprises a series of events about the fluid sample. Each event comprises a plurality of parameter values. Each of the plurality of the parameter values is associated with one parameter from a plurality of parameters. The plurality of parameters comprises scatter parameters and antigen parameters. The computing system operates upon the one or more cytometry datasets. Each marker pair comprising a first parameter and a second parameter. The first parameter and the second parameter are selected from the plurality of parameters. The area comprises units, each of the units having an x-unit range and a y-unit range. The units comprise cluster units and empty units. Each of the one or more event populations comprise one or more clustered events from the series of events. For each marker pair the computing system is configured to: (1) for each of the one or more event populations, assign each of the clustered events to one of the cluster units based on the parameter value for the first parameter of each clustered event, the parameter value for the second parameter of each clustered event, the x-unit range of each cluster unit, and the y-unit range of each cluster unit; each cluster unit comprises at least one clustered event and a cluster event density representative of the number of clustered events in the cluster unit, (2) determine the event population for each of the cluster units from the one or more event populations based on one or more of i) the cluster event density, ii) a closest cluster population, iii) a distance to closest population, and iv) one or more intervening density differences, (3) determine an immunophenotype for each event population based on one or more of i) an antigen median fluorescence intensity for each antigen parameter represented in the event population, ii) an antigen parameter expression, iii) an antigen parameter intensity, and iv) a light chain expression, (4) determine one or more population classifications based on the immunophenotype for each event population, and (5) generate a diagnostic report based on the one or more population classifications.

In another embodiment of the present invention, each event represents a cell in the fluid sample. Each of the one or more event populations comprises a population size greater than a minimum analyzable population size. The x-axis comprises an X-count unit and the y-axis comprises a Y-count unit. Each clustered event is assigned to the one cluster unit if the parameter values of the clustered event are within the x-unit range and y-unit range of the cluster unit. The computing system sorts the plurality of the cluster units according to the cluster event density of each cluster unit.

In yet another embodiment of the present invention, each antigen parameter is associated with a parameter cutoff value. Each of the one or more event populations comprises a quantity of marker pair positive events. The quantity of marker pair positive events is greater than a minimum event count. Each of the marker pair positive events comprises the parameter value from the plurality of parameter values greater than the parameter cutoff value. The parameter value is one of i) the first marker pair parameter value and ii) the second marker pair parameter value.

In another embodiment of the present invention, the distance to closest population represents the distance that is shortest between the cluster unit and a closest assigned cluster unit from the plurality of cluster units. The closest assigned cluster unit comprises the closest cluster population. The closest cluster population is selected from the one or more event populations. The event population is the same as the closest cluster population if the distance to closest population is less than a same population distance limit. The event population is a new subpopulation if the distance to closest population is greater than a new population distance limit. The computing system is configured to determine the event population based on the intervening density differences between the cluster unit and one or more intervening cluster units if the distance to closest population is between the new population distance limit and the same population distance limit.

In yet another embodiment of the present invention, the apparatus further comprises the one or more intervening cluster units. The one or more intervening density differences are density differences between the cluster unit and each of one or more evaluated intervening cluster units. The one or more evaluated intervening cluster units are selected from among the one or more intervening cluster units.

In another embodiment of the present invention, each of the intervening density differences is associated with one of the evaluated intervening cluster units and with an associated intervening density difference cutoff. The event population is the closest cluster population if each of the intervening density differences is less than or equal to its associated intervening density difference cutoff. The event population is the new subpopulation if at least one of the intervening density differences is greater than its associated intervening density difference cutoff.

In yet another embodiment of the present invention, the x-unit range of each intervening cluster unit is between the x-unit range of the cluster unit and the x-unit range of the closest assigned cluster unit. The y-unit range of each intervening cluster unit is between the y-unit range of the cluster unit and the y-unit range of the closest assigned cluster unit. The one or more evaluated intervening cluster units are selected from the group consisting of i) all intervening cluster units, ii) intervening cluster units that are crossed by a straight line connecting the cluster unit and the closest assigned cluster unit, and iii) one or more randomly selected evaluated intervening cluster units from among the one or more intervening cluster units.

In another embodiment of the present invention, the area is a square and comprises an equal number of units along the x-axis, and along the y-axis.

In yet another embodiment of the present invention, the area comprises 400 units.

In another embodiment of the present invention, each of a plurality of antigen expressions is determined based on an antigen MFI value for each of the antigen parameters in the event population. Each of the plurality of antigen expressions is selected from a positive antigen expression, a negative antigen expression, and an equivocal antigen expression. Each of a plurality of antigen parameter intensities is determined based on the antigen MFI values for an antigen parameter having the positive antigen expression. Each of the plurality of antigen parameter intensities is selected from a dim intensity, a moderate intensity, and a bright intensity.

In yet another embodiment of the present invention, the event populations further comprise a B-cell population. The B-cell population comprises positive antigen expressions for B-cell parameters. The B-cell parameters are selected from the group consisting of CD19, CD20, CD22, CD79a, CD79b, and combinations thereof. The computing system is configured to determine one or more light chain expression ratios for each of the B-cell populations based on a lambda parameter value and a kappa parameter value.

In another embodiment of the present invention, each of the one or more event populations comprises a population size ratio. Each population size ratio is representative of a ratio of each event population size to the number of events in the series of events.

In yet another embodiment of the present invention, the diagnostic report is generated based on one or more of i) population classifications, ii) population immunophenotyping, iii) light chain expression ratios, and iv) population size ratios.

In another embodiment of the present invention, the B-cell parameters are further selected from the group consisting of CD24, CD27, PARS, OCT2, BOB1, immunoglobulin, and combinations thereof.

### BRIEF DESCRIPTION OF THE SEVERAL VIEWS OF THE DRAWING

The advantages and features of the present invention will be better understood as the following description is read in conjunction with the accompanying drawings, wherein:

FIG. 1 is a diagram illustrating an embodiment of the present invention.

FIG. 2 is a diagram of a flow cytometry dataset from an embodiment of the present invention.

FIG. 3 is a diagram illustrating a data cleaning embodiment of the present invention.

FIG. 4 is a diagram illustrating a clustering process of an embodiment of the present invention.

FIG. 5 is a diagram illustrating a clustering process of an embodiment of the present invention.

FIG. 6 is a diagram illustrating a coordinate system of an embodiment of the present invention.

FIG. 7 is a diagram illustrating an embodiment of the present invention.

FIG. 8 is a diagram illustrating an embodiment of the present invention.

FIG. 9 is a diagram illustrating an embodiment of the present invention.

FIG. 10 is a diagram illustrating a report generated by an embodiment of the present invention.

For clarity all reference numerals may not be included in every figure.

### DETAILED DESCRIPTION OF THE INVENTION

Embodiments of the present inventions can be used to automate the process of flow cytometry data collection, interpretation, analysis, and diagnosis, and can improve the standardization, diagnostic accuracy, and efficiency of flow cytometry diagnostics. Embodiments of the present invention may be particularly helpful for leukemia/lymphoma evaluation.

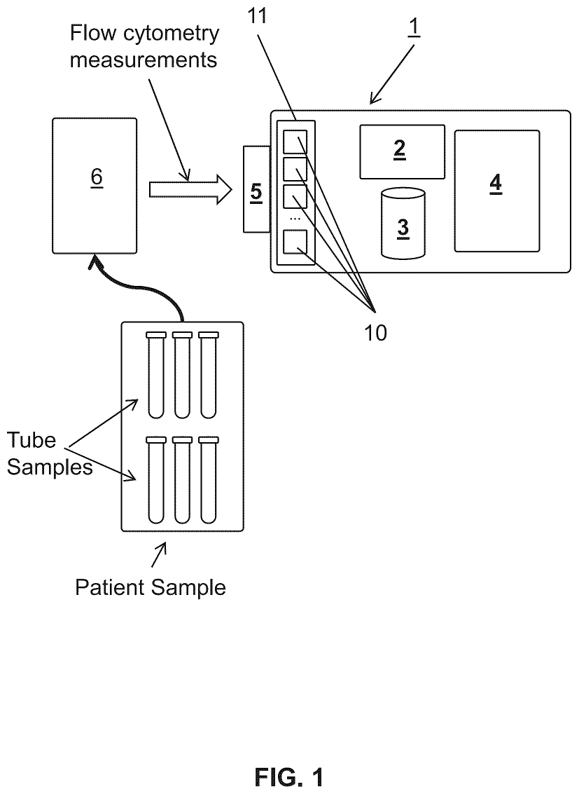

An exemplary system 1 according to the present invention, as illustrated in FIG. 1, may comprise a computing system 2 that operates on one or more flow cytometry datasets 10, also referred to here as just datasets 10. The computing system 2 may be coupled to one or more of an input device 5, a display, and a datastore 3. The computing system 2 and/or datastore 3 may also be communicatively connected (via wired or wireless, e.g., network; direct data connection, etc.) to a flow cytometer 6, a central data repository, a telehealth system, and various other systems and modules for the provision of diagnostic and/or healthcare services. System 1 may further comprise a user interface 4 coupled with the computing system 2 and the display.

Flow cytometer 6 may generate flow cytometry measurements in the form of a dataset 10 for a sample tube (e.g., a test tube containing a fluid sample) run through the cytometer 6. Datasets 10 may be in various data formats, but preferably are organized according to the Flow Cytometry Standard (“FCS”) format developed and maintained by the International Society for Advancement of Cytometry. Datasets 10 in any other format, such as plain text, xml, encrypted, binary, may also be analyzed using the present invention as will be understood by persons skilled in this field.

Dataset 10 may comprise flow cytometry information about the fluid sample in a sample tube, preferably represented as a series of events (or rows) of data.

A fluid sample from a patient may be separated into several sample tubes and each tube tested in a flow cytometer 6. Dataset 10 may include cytometry information about one or more sample tubes. When cytometry information about a patient is divided into more than one patient datasets 10, the present invention may be configured to perform analyses on each of the patient datasets 10 to assign events 20 to one or more event populations 100 (also referred to here as populations), and to combine event populations, classifications, and other diagnostic parameters determined from each of the patient's datasets 10 in order to obtain a clearer diagnostics and analysis based on a complete patient sample.

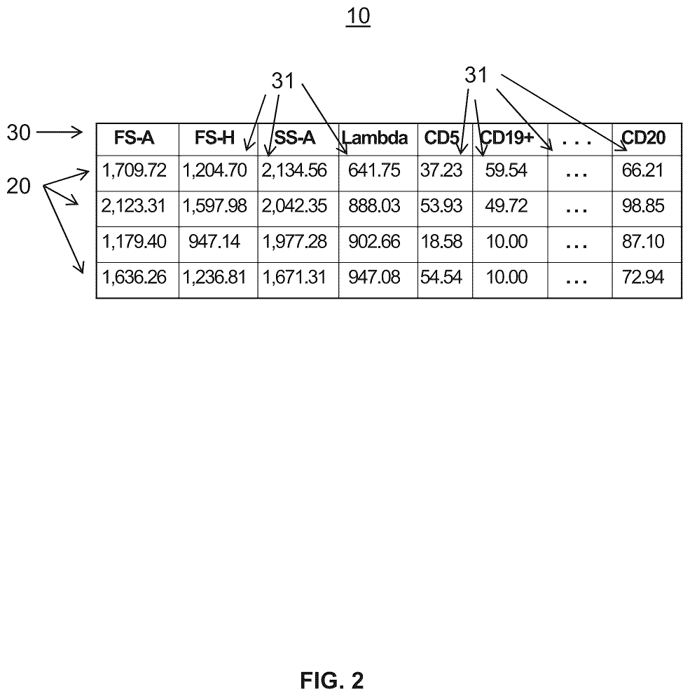

Each event 20, illustrated as a row in FIG. 2, in a dataset 10 may represent a cell from a sample tube, and each event 20 (e.g., cell) is associated in the dataset 10 with a plurality of parameter values 31, illustrated as columns in the table of FIG. 2. Each parameter value 31 may be a digital representation of a fluorescence value recorded by flow cytometer 6, corresponding to a parameter 30. Parameters 30 are exemplified in the first row of the table in FIG. 2. Parameter values 31 may represent the wavelength, photon energy, frequency, or other fluorochrome indicators; or light scatter characteristics of cells. Parameters 30 whose presence may be tested in a flow cytometer 6 and recorded in dataset 10 may be extensive and may comprise various antigen parameters, thousands of which are well known in the medical field with new ones continuously being discovered, light scatter parameters 30 (e.g., forward, side), and other parameter types.

Parameter values 31 in dataset 10, preferably are represented as positive numerical values but in certain situations may also be negative. Parameter values 31 may have varying precisions and wide value ranges depending on the parameter, fluorochrome, equipment, calibration, environmental conditions, type of sample, and various other factors. Antigen parameter value 31 associated with an event (e.g., a cell) may be representative of the amount of antigen present in the event (i.e., the cell). Scatter parameter values 31 generally may be representative of physical characteristics of a cell represented by an event (e.g., forward scatter may correlate with cell size, side scatter may correlate with cell complexity).

Embodiments of the present invention may receive datasets 10 and store them on datastore 3 (e.g., disk, database, memory, cache, etc.). Datasets 10 may be received in various ways, for example, through an input device 5, such as a user input device 5 (e.g., scanner, external memory, flash drive, keyboard, voice input, etc.) or an input port 5 (e.g., for wired or wireless networking, point to point transmission, etc.), and through various other methods. Datasets 10 may be received from flow cytometer 6, from a remote system or device (not shown), a central healthcare related system (including, e.g., data repository), and various other sources.

Depending on the format, condition, ranges, data standard, and other factors, various data preparation actions, such as data extraction, data source compensation, data cleaning, data transformation, and others, may need to be performed on datasets 10 that will result in data in datasets 10 better suited for analysis. For example, a parameter value 31 extraction may be performed to remove certain unnecessary data from the datasets 10 (or to extract parameter values), so that datasets 10 comprise parameter values 31 and other information needed for the analysis.

Data Source Compensation may be performed on the parameter values 31 for various reasons, for example, if the datasets have been obtained with different flow cytometers 6, received from different data sources, or subject to other factors or circumstances. Datasets 10 may be received in the datastore after having undergone data source compensation, or they may not need data source compensation. Embodiments of the present invention may be configured to apply data source compensation to the parameter values 31 in the datasets 10. Further, computing system 2 and/or datasets 10 may be configured to enable querying if data source compensation is needed, or should be performed. For example, when datasets 10 are provided as FCS files, parameter values 31 may be compensated by inverting a spillover matrix provided in the FCS file to obtain compensation values and multiplying the parameter values 31 by the compensation values.

Embodiments of the present invention may also perform data transformations (including data ranging) to confine the parameter values 31 to a desired analytical data range if the Datasets 10 received in datastore 3 contain parameter values 31 outside of that analytical range. For example, if an analytical data range has a minimum value (e.g., zero for a positive analytical data range) the parameter values 31 in the received datasets 10 that are below the minimum value (e.g., negative) may be transformed (e.g., “moved,” recalculated) to the analytical data range while maintaining the relative distribution of the parameter values 31 from their original value range. In situations where the analytical data range has a maximum value upper limit, parameter values 31 exceeding the maximum value can be transformed to fit within the upper limit (e.g., by recalculating and evenly distributing the parameter values 31 within the data range expected for that parameter). Alternatively, parameter values 31 exceeding the upper limit may be excluded from the datasets 10 if their contribution may be insignificant.

Embodiments of the present invention may also perform data cleaning of the parameter values 31 in datasets 10, by removing debris, data doublets, events with abnormally high scatter parameter values 31, and other artifacts or non analytical data.

Data may be cleaned from debris by removing from datasets 10 events having a scatter parameter value below a debris scatter cutoff, which may be predetermined, or calculated based on the data in the datasets. The debris scatter cutoff may depend on the flow cytometer calibration (e.g. voltages corresponding to parameter values 31), may be provided for a particular cytometer, or may be based on a calibration. As an example, conventionally the debris scatter cutoff may be determined by visual analysis of scatter plots of normal leukocyte populations, where the debris scatter cutoff is the point below which normal leukocytes are not seen or are seen in negligible numbers. The debris scatter cutoff can also be determined statistically, for example by setting it at the level above which a high percentage (e.g., 95% or 99%) of normal cell (e.g., lymphocyte) scatter measurements occur. The scatter values and scatter cutoffs described above, preferably are forward scatter.

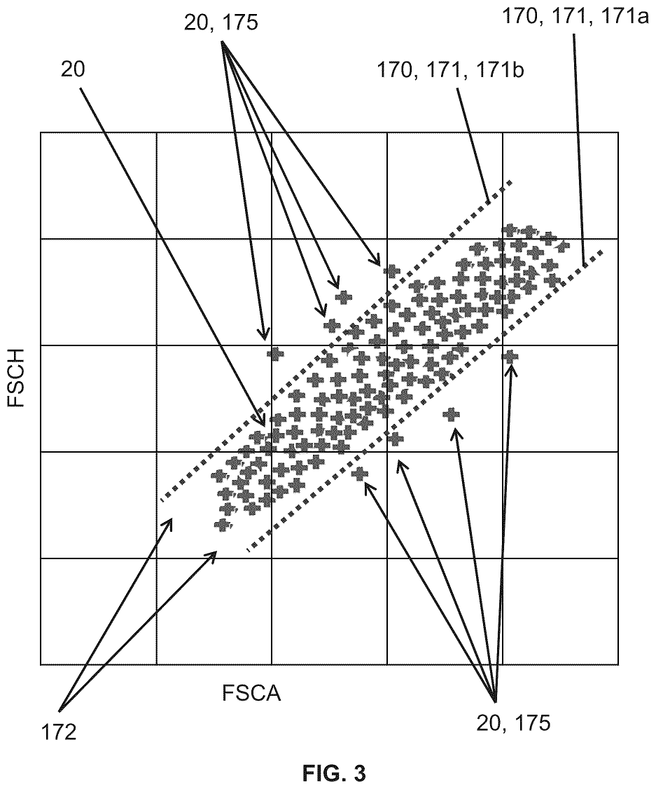

Data doublets 175 may result from flow cytometer 6 simultaneously recording parameter values for two cells, instead of one cell (e.g., when two cells pass together). Data Doublets 175 may be removed from the datasets 10 by, for example, as illustrated in FIG. 3, using functions 170 for single cell lines 171 on a plot of forward scatter area parameter values (indicated as “FSCA”) vs. forward scatter height parameter values (indicated as “FSCH”) to define a single cell data envelope 172. Functions 170 may be of any order, but preferably functions 170 may be linear functions 170 for a first straight line 171a (e.g., a minimum single cell straight line 171a) and a second straight line 171b (e.g., a maximum single cell straight line 171b) that approximately correspond to the limits (or envelope boundaries) of scatter parameter values 31 that can be recorded for single cells. Higher order functions 170 and curves 171 may be used if desired. Linear functions 170 for lines 171 illustrated in FIG. 3 as thick dotted lines, may be as follows:

First straight line 171a: FSCH=A+B*FSCA

Second straight line 171b: FSCH=C+D*FSCA

Computing system 2 may receive function parameters A, B, C, and D through the user interface 4, together with datasets 10 (e.g., based on previous statistical analysis), from local or remote data storage, and various other methods and/or sources. Computing system 2 may also calculate the function parameters based on statistical analysis of datasets 10. Events 20 with parameter values 31 for FSCH and/or FSCA outside data envelope 172 may be removed as data doublets 175. Events 20 having a maximum forward scatter parameter value (e.g. the highest value in a dataset) may also be removed depending on the scatter voltage settings, as these events may contain data artifacts and non analyzable data. Preferably, during data cleaning the computing system 2 may be configured to keep track of the removed debris count, removed data doublets count, and removed events count.

Dataset quality control may also be performed according to the current invention. For example, file names, properties, metadata, checksums, hashes, and other information of multiple datasets 10 may be compared to ensure consistency, that all the data belong to the same fluid sample, or come from the same patient. In another example, anomalies in acquisition, such as air bubbles, may be detected by examination of one or more parameter values 31 (e.g., side scatter) over time. For Dataset quality control of an assessed maker, a control time unit of certain duration maybe chosen, and the parameter values 31 for the assessed parameter may be analyzed (e.g. compared, evaluated) for a period of one or more control time intervals for which assessed parameter values 31 are available. A significant change between control time intervals in the number of events in data set 10, or in the Median (or Mean) Fluorescence Indication (“MFI”) of the assessed parameter values 31, may indicate a potential anomaly during acquisition and an alert may be generated indicating that analysis should not continue due to suboptimal data quality. When multiple datasets 10 need to be analyzed, parameter values 31 of common parameters 20 that are common to two or more of the multiple datasets 10 may be analyzed to detect differences that may be indicative of suboptimal data quality. Differences between parameter values 31 for the same parameter 20 in different datasets 10 may be displayed on user interface 4 for a user to determine data quality, or alternatively, certain data quality cutoffs may be provided (e.g., as data quality variables, hardcoded, etc.) and when data quality cutoffs are exceeded certain actions may be taken, for example, an alert generated, suboptimal data excluded, and others. For example, if the MFI of a parameter, or the percentage of events having parameter values exceeding certain levels, differ between two datasets 10 and the difference exceeds a data quality cutoff, that may be indicative of suboptimal data quality and generate an alert. MFI values may be determined using median, arithmetic mean, or geometric mean of parameter values 31, or through other statistical methods.

Embodiments of the present invention may be configured to apply isotype control to datasets 10 based on isotype control values that may be provided in a separate isotype control file, a control dataset, or otherwise. For example, computing system 2 may be configured to compare parameter values 31 in the datasets 10 to the isotype control values and apply isotype control based on that comparison. Isotype controls may be applied in various ways, but preferably, the isotype control values for each parameter may be subtracted from the corresponding parameter values 31 in the datasets 10.

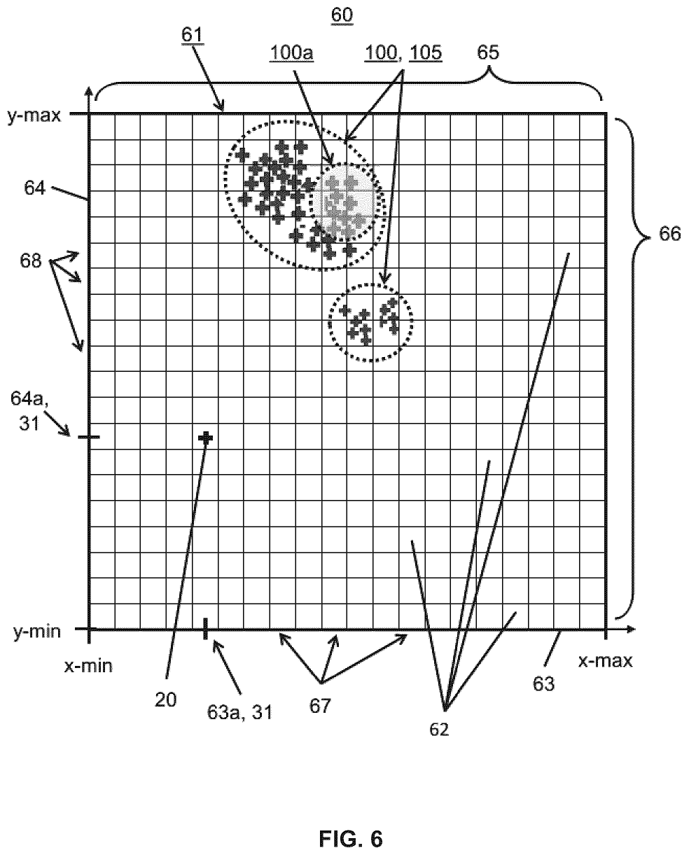

Embodiments of the invention may represent events 20 from a dataset 10 as bivariate data where a first parameter variable 63 (e.g., x-parameter variable 63, plotted along the x-axis) is paired with a second parameter variable 64 (e.g., y-parameter variable 64, plotted along the y-axis). FIG. 6, visualizes a bivariate cartesian coordinate system 60 comprising 400 (20 by 20) units 62. An event 20 may be plotted according to a first coordinate 63a corresponding to a first parameter value 31 of the first parameter variable 63 and a second coordinate 64a corresponding to a second parameter value 31 of the second parameter variable 64. Parameter variables 63, 64 may be paired as marker pair parameters 63, 64, and may be represented as coordinate parameter variables 63, 64 (e.g., x- and y-parameter variables). The computing system 2 may represent the coordinate system as a data structure. The area 61 of the bivariate coordinate system 60 may be divided into units 62, preferably rectangles, and more preferably squares, but units 62 may be other geometrical shapes. The X-range (e.g., indicated as x-min to x-max) of x-parameter values 31 along the x-axis 63, Y-range (e.g., indicated as y-min and y-max) along the y-axis 64, and the number of units 20 (e.g., area 61) may be received or obtained through, or from, various methods and sources, for example, through input device 5, stored locally on the datastore or remotely, hardcoded, calculated by the computing system 2, and various others. For example, the X-range and the Y-range may be based on the full range of parameter values 31 of the x-parameter variable 63 and the y-parameter variable 64 in the dataset 10. The X-range and the Y-range may also be set large enough to encompass all parameter values 31 for any x-parameter variable 63 and the y-parameter variable 64, eliminating the need to calculate the X- and Y-ranges for each pair of x- and y-parameter variables 63, 64 represented in the datasets 10. The X-range and the Y-range may also be set to other values that are determined to facilitate the analysis.

Embodiments of the invention may utilize a rectangular coordinate system 60 having an X-count 65 number of units 62 along the x-axis, and a Y-count 66 number of units 62 along the y-axis. FIG. 6 illustrates a coordinate system 60 wherein X-count 65 and Y-count 66 are each 20. Coordinate system 60 may comprise an area 61 comprising a number of units 62 equal to the product of X-count 65 and Y-count 66. Each unit 62 may be a rectangle, a square, or another geometric shape, and all units 62 may be equal in size, and/or area. The X-count 65 and Y-count 66 units may be evenly distributed along the x- and y-axes, respectively. The x-axis and y-axis may utilize a linear scale (preferred, e.g., for scatter parameter values 31) or a logarithmic scale (preferred, e.g., for antigen parameters 30).

The invention is described herein using a preferred embodiment utilizing a rectangular coordinate system comprising 20 units along the x-axis, 20 units along the y-axis, and an area of 400 units. For readability, the figures illustrate a portion of the preferred embodiment coordinate system. References to, or illustrations of, a particular coordinate system or size thereof, and square units, should not be understood to be limiting and are used for illustration purposes.

An event population 100 (indicated as dotted ovals in FIG. 6) is a grouping of events 20 (e.g., representing fluid sample cells) that show similar characteristics in a flow cytometry testing. Events in a population have one or more parameters with similar parameter values 31 and that may cause the events to appear as a distinct cluster on a scatter plot. Population 100 has a population size 105 which preferably is representative of the number of events in the event population 100.

In situations where events are assigned to, for example, an initial population 100, all events in initial population 100 may be analyzed according to the present invention to determine if any events 20 in the initial population 100 may be assigned to a new population 100, which may be a subpopulation 100a (shown as a shaded oval in FIG. 6) of the initial population 100. As explained in more detail below, the events 20 in the new population 100 may also be analyzed to determine if any events 20 may be separated into another new subpopulation 100b of the new population 100. The present invention allows the analytical granularity, accuracy, speed, diagnostics, and other characteristics of the analyses according to embodiments of this invention, to be affected and/or controlled, by enabling the use of various limits or cutoffs 91, such as a minimum analyzable population size 92, a minimum new subpopulation size 93, minimum event count 95, parameter cutoff value 94, and others. By way of an example, events in an event population 100 (e.g., initial population, new population, subpopulation) may be analyzed for the presence of a subpopulation 100 if the number of events in the event population exceeds the minimum analyzable population size 92 (e.g., 20 events). In another example, a group of events that could be separated into a subpopulation, will be assigned to a new event population only if the number of such events exceeds the minimum new subpopulation size 93 (e.g., 30 events). Limits 91, such as minimum analyzable population size 92, minimum new subpopulation size 93, minimum event count 95, parameter cutoff value 94, and others, may be provided by or through the user interface, may be hardcoded, or may be determined by the computing system 2. For example if a set of population limits does not produce a satisfactory result, a different set of population limits may be provided or determined and analyses performed with them.

Embodiments of the present invention may comprise a set, or a plurality, of marker pairs 50 which may be stored in the datastore, hard coded, determined by the computing system 2 based on the parameters 30 represented in datasets 10, or received from external source through the input device 5 (or port) and/or user interface 4. Each marker pair 50 comprises a marker pair first parameter 63 and a marker pair second parameter 64, which may be selected from the parameters 30 represented in datasets 10. Each marker pair parameter 63, 64 may be a marker pair antigen parameter 30, or a marker pair scatter parameter 30. The marker pairs 50 preferably should include all parameters that could potentially show parameter differences between event populations. Detection of such parameter differences may facilitate the identification of distinct populations 100.

Embodiments of this invention may be configured to analyze the events 20 of each analyzable population 100 based on each marker pair 50 (by, e.g., analyzing the events based on the parameters values for the first and second parameters 63, 64 of the marker pair 50). In some embodiments, an analyzable population 100 for a marker pair 50 may be a marker pair analyzable population 100 having more than a minimum event count 95 of marker pair positive events 20 for the marker pair 50. Marker pair positive events 20 may be events 20 having a parameter value 31 exceeding a parameter cutoff value 94 for at least one of the marker pair parameters 63, 64. A parameter cutoff value 94 may be associated with each antigen parameter. For example, a parameter cutoff value 94 may have a value of 10, and the minimum number of events exceeding the parameter cutoff value may have a value of 15. If at least 15 events exceed the cutoff of 10 for at least one parameter in the marker pair, the marker pair 50 is analyzed as a representative marker pair. And if there are less than 15 events exceeding the cutoff of 10, the marker pair 50 is not analyzed.

Computing system 2 may be configured to perform a clustering process 500, for each marker pair 50 (or each represented marker pair 50 in event 20), and recursively for each population (including for the initial population, and for each new population 100a and for each subpopulation 100 that may be identified during process 500) analyze each event 20 based on the parameter values for each first parameter variable 63 and second parameter variable 64, identify new populations 100, and subpopulations, and assign each event to an event population which may be selected from the initial population, the new populations, and the new subpopulations 100a. The computing system 2 may be further configured to repeat the analysis of each event for each marker pair 50, and based on newly identified event populations, until no new populations can be identified, and all events have been assigned to an event population. In some embodiments, the analysis of each event 20 may be repeated only for each marker pair 50 that is represented in event 20 (a represented marker pair 50).

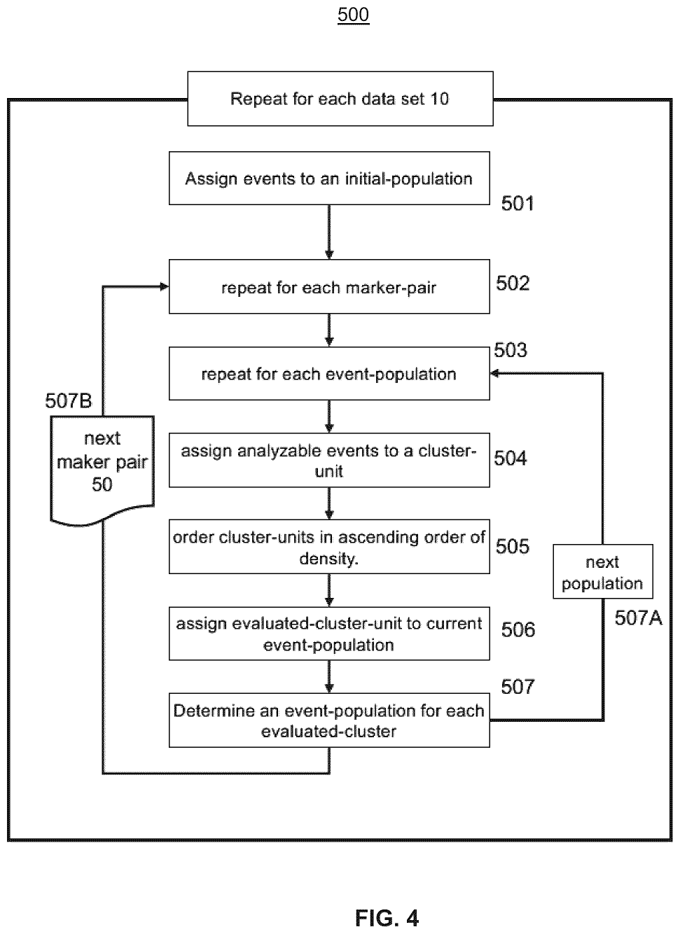

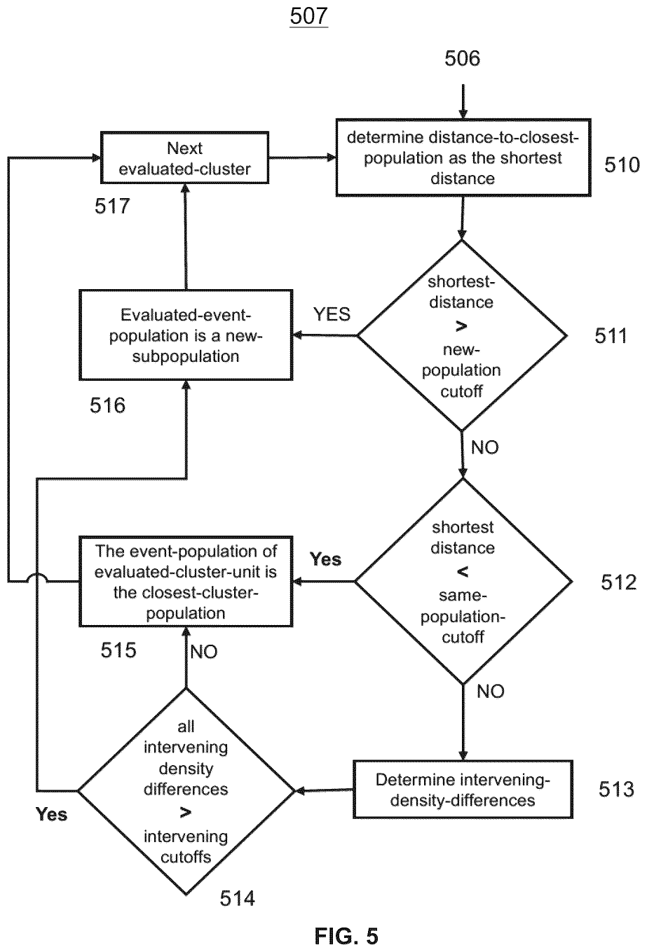

An example of a clustering process 500 according to one embodiment is illustrated in FIGS. 4, 5. Clustering process 500 may begin an analysis of a new dataset 10 in step 501 by assigning all events 20 to an initial population 100. Process 500 may then proceed to step 502, beginning with the first marker pair 50, to analyze all events, based on parameter values 31 of the first parameter variable 63 and second parameter variable 64.

By way of example, in step 502, computing system 2 may generate a data structure representing a bivariate coordinate system 60 having an area of N by M units (e.g., N and M corresponding to X-count 65 and Y-count 66, respectively), an x-axis 63 and a y-axis 64. The x-axis may comprise an x-range having an x-max value and an x-min value and the y-axis may comprise a y-range having an y-max value and a y-min value. The x-axis may be divided into N equal x-ranges 67, and the y-axis may be divided into M equal y-ranges 68, so that each of the N by M units may be represented as having an x-unit range 67, or a unit-x-coordinate 67, and a y-unit range 68 or a unit-y-coordinate 68, as illustrated in FIG. 6. Each of the N by M units can be a cluster unit 62a having been assigned one or more events 20 or may be an empty unit 62b. Computing system 2 may generate a coordinate system 60 (represented, e.g., by a data structure in computer memory) for each marker pair 50, or may generate one coordinate system to be used for all marker pairs 50.

In the same example, in step 503, for each event population each event 20 in (or associated with) the event population is clustered in (i.e., assigned to) a cluster unit 62a as a clustered event 20 based on the parameter value 31 for the First parameter variable 63 of the clustered event, the parameter value 31 for the second parameter variable 64 of the clustered event, the x-unit range 67 of each cluster unit, and the y-unit range of each cluster unit 68. The events are assigned to a cluster unit 62a as a clustered event based on the parameter values 31 for the First parameter variable 63 and Second parameter variable 64 being within the x-unit range and the y-unit range of the cluster unit.

In steps 505 through 507 the computing system 2, as illustrated by process 500, is configured to evaluate each cluster unit 62a and determine an event population for each evaluated cluster unit 62c and for the associated clustered events 20, by determining whether the clustered events in an event population 100 may be subdivided into distinct subpopulations 100. The computing system 2 may determine the event population for each evaluated cluster unit 62a based on one or more of the cluster event densities, a closest cluster population 100, a shortest distance 97 to cluster unit population, and one or more intervening density differences. In some embodiments shortest distance 97 may be the distance to the closest cluster population 100 or distance to the closest population 100.

When all events from the event population are assigned to one or more clustered units 62a as clustered events 20, as illustrated in Step 504, the computing system 2 may determine the cluster unit density 22 of each cluster unit. The cluster unit density 22 may be representative of the number (e.g., count) of clustered events in the cluster unit. In optional step 505, the cluster units may be sorted according to the cluster event density 22 of each cluster unit (e.g., ascending, or descending order), and the cluster unit with highest density (e.g., the most events) is analyzed as an evaluated cluster unit 62c.

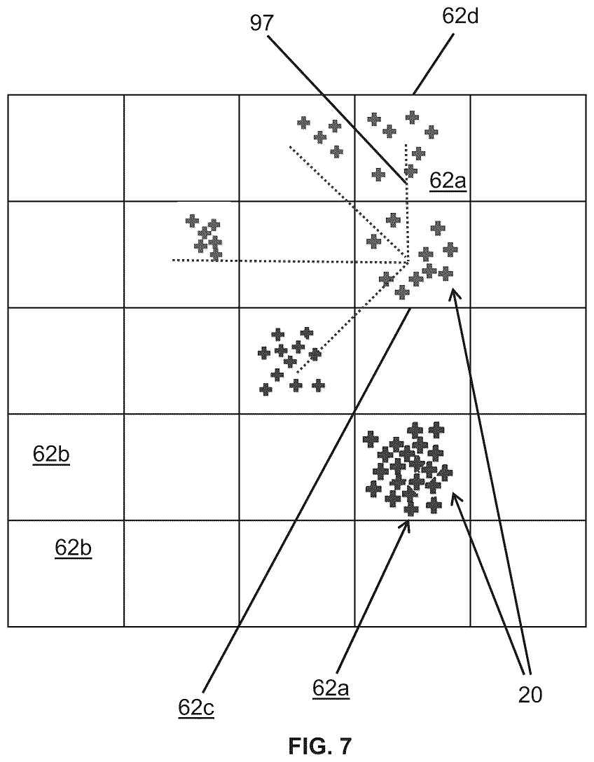

To determine the event population of an evaluated cluster unit 62c the computing system 2 may first assign, in step 506, the evaluated cluster unit 62c to the current event population (e.g., initial population in a new dataset, a previously identified event population) and then in step 507 perform an event population analysis of evaluated cluster units 62c. In step 507, the computing system 2 may determine the distances between an evaluated cluster unit 62c and other cluster units 62a already assigned to event populations 100. In step 510, as illustrated in FIG. 7, the shortest distance among all determined distances is selected as a shortest distance 97 to a cluster unit 62a having an event population 100. The closest cluster unit 62d located at the shortest distance 97 may comprise a closest cluster event population 100.

In steps 511-517 The computing system 2 may a) assign the closest cluster population as the evaluated cluster event population if the shortest distance 97 to closest population is less than a same population distance limit, as illustrated in steps 511, 516; b) determine that the evaluated cluster event population may be a new subpopulation if the shortest distance 97 is greater than a new population distance limit, as illustrated in steps 512, 515; and c) if the shortest distance 97 is between the new population distance limit and the same population distance limit the computing system 2 may be configured in step 513 to determine the evaluated cluster event population based on the intervening density differences between the evaluated cluster unit and one or more evaluated intervening cluster units 112 by performing an intervening densities analysis. Each evaluated intervening cluster unit 112 may have an associated intervening density difference cutoff 116.

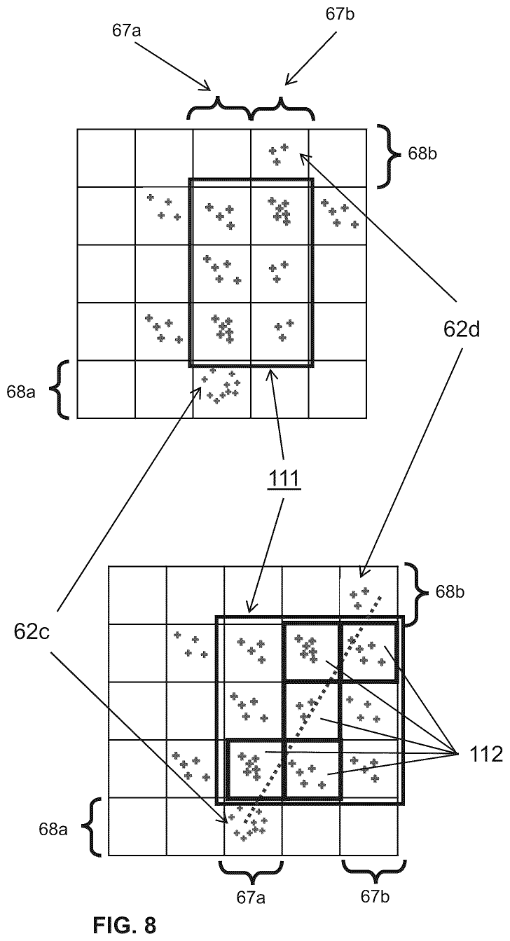

For an intervening densities analysis in step 513, as illustrated in FIG. 8, computing system 2 may be configured to identify one or more intervening cluster units 111 each having an intervening unit 111 x-unit range 67 between the evaluated cluster unit x-unit range 67a of the and the closest assigned cluster x-unit range 67b of the closest assigned cluster, and an intervening unit y-unit range between the evaluated cluster unit y-unit range 68a and the closest assigned cluster y-unit range 68b. In FIG. 8, intervening cluster units 111 are illustrated by being within a thick rectangle. The computing system 2 may select one or more evaluated intervening cluster units 112 and determine one or more intervening density differences between the evaluated cluster unit density and densities of each of one or more evaluated intervening cluster units. The evaluated intervening cluster units 112 may include i) all intervening cluster units 111, ii) intervening cluster units 111 that are crossed by a straight line connecting the cluster unit and the closest assigned cluster unit, and iii) one or more randomly selected evaluated intervening cluster units 112 from among the one or more intervening cluster units.

The computing system 2 may a) assign the closest cluster population as the event population of the evaluated cluster if each of the intervening density differences is less than or equal to its associated intervening density difference cutoff, or b) determine that the event population of the evaluated cluster may be a new subpopulation if at least one of the intervening density differences is greater than its associated intervening density difference cutoff;

For each marker pair, the clustering analysis 500 (to determine an event population for the events in each cluster unit) repeats for all events assigned to an analyzable population 100. Embodiments may be configured to perform clustering analysis 500 of events 20 in all analyzable populations 100, or of events 20 in analyzable populations 100 having a population size 105 greater than a minimum analyzable population size 92. During clustering analysis 500, computing system 2 may assign cluster units 62 (e.g., events 20 in cluster units 62) to a new event population 100a based on a new population or a new subpopulation (population or subpopulation, e.g., newly identified during analysis) if the new population or new subpopulation have a population size 105 greater than the minimum new subpopulation size 93. Process 500 analysis may proceed by evaluating one cluster unit 62a at a time wherein the evaluated cluster unit 62a is selected based on the density 22 of cluster units 62a, starting with the cluster unit 62a having the highest density 22 and ending when an empty unit 62b is reached. The analysis then proceeds to the next event population 100, as illustrated by step 507A, and repeats to evaluate each cluster unit (and each clustered event therein) in the next event population until all clustered events in cluster units having been assigned an event population 100 have been evaluated. The analysis then repeats for each marker pair as illustrated by step 507B.

Embodiments of computing system 2 may be configured to determine an immunophenotype 125 for each population 100. An immunophenotype 125 for a population 100 (or a population immunophenotype 125) may be an initial immunophenotype 125, a full immunophenotype 125, and a final immunophenotype 125, determined at different analysis stages, as illustrated below. For example, an Initial immunophenotype 125 may be determined for each population 100 in each dataset 10 following clustering process 500, a full immunophenotype 125 may be on each population of a combined dataset 11, and a final immunophenotype 125 may be determined on each population following further analysis of the combined dataset 11 as described below, to improve diagnostic accuracy.

For example, computing system 2 may determine an immunophenotype 125 for each event population 100 based on one or more of i) an antigen Median (or Mean) Fluorescence Intensity (“antigen MFI”) for each antigen parameter 30 represented in an event population 100, ii) an antigen parameter expression, iii) an antigen parameter intensity, and iv) a light chain expression 124.

One or more antigen MFIs for population 100, may be calculated for each antigen parameter 30 based on the parameter values 31.

Population antigen MFIs may be used to determine population antigen parameter expressions (interchangeably referred to as an antigen expression or a parameter expression) for each antigen parameter 30 represented in an event population. For example, values of population antigen MFI above a positive expression cutoff value (e.g., 300) may be marked as positive expression status, below a negative expression cutoff value (e.g., 100) may be marked as a negative antigen expression status, and antigen MFI between the positive and negative expression cutoff values may be marked as an equivocal antigen expression status. parameter/Antigen expression status may also be determined based on a percentage of events 20 exceeding a fluorescence cutoff value. For example, if 20% of events in an event population exceed a fluorescence value for an antigen parameter 30, then the parameter's expression status in that population may be labeled as positive. If 10% to 20% of events in a population exceed the fluorescence cutoff value for that parameter, then the antigen's/parameter's expression status in that population may be labeled as equivocal, and if less than 10% of events exceed the fluoresce cutoff value, then parameter's expression status in that population may be labeled as negative.

Population parameter intensity levels (e.g., dim, moderate, or bright) may also be determined for each parameter in a population having a positive population antigen/parameter expression for that parameter. Population parameter intensity level may be defined based on value ranges of each population antigen MFI for that parameter. For example, MFI values above 1,000 may be labeled as bright, between 100 and 1000 may be labeled as moderate, and between 10 and 100 may be labeled as dim. The 10, 100, and 1000, are provided as illustrative examples; the actual values may widely vary depending on many factors, including for example, parameter values 31 in a dataset, flow cytometer 6, cytometer calibration, data ranges, and various other factors.

Event population identified as B-cell populations comprising positive antigen expressions for B-cell antigen parameters 30 (e.g., CD19, CD20, CD22, CD79a, CD79b, CD24, CD27, PAX5, OCT2, BOB1, and immunoglobulin) may undergo further analysis. For example, for each B-cell population the computing system 2 may be configured to determine a population light chain expression 124 based on the parameter values 31 of Lambda and Kappa parameters for each event and the ratios of Lambda and Kappa parameter values 31. The proportion of cells expressing light chains is determined based on the percentage of cells expressing either kappa or lambda. If this percentage of cells is too low, it can indicate that lymphoma is present, similar to how an altered kappa to lambda ratio can indicate lymphoma. B-cell population Kappa Lambda light chain ratios 124 may be determined, for example through a binomial proportion confidence interval (e.g., a Wilson score interval), another statistical confidence interval, or using other methods. Kappa Lambda ratios may be classified as normal, abnormal, or light chain excess. Particularly large or small values for Kappa Lambda ratios indicating that one type of light chain is significantly more common than the other are considered abnormal and may indicate a malignant condition (e.g., B-cell lymphoma). For example, Kappa Lambda ratios well outside of certain limits (e.g., 3:1 and 0.3:1) may be considered abnormal and may indicate the presence of lymphoma, while Kappa Lambda ratios that are well within the limits may indicate normal ratios. Such limits are determined from clinical and empirical research and medical practice, and from analyzing samples of B-cell populations. Alternatively, Kappa Lambda ratios 124 that cannot definitively be defined as normal or abnormal are referred to as light chain excess indicators. For example, a ratio of 3:1 (three-to-one) may be considered a light chain excess indicator, as it is a borderline between normal and abnormal, and cannot solely be used to determine the presence or absence of B-cell lymphoma.

Embodiments of the present invention may also be configured to determine one or more population classifications 121 based on the one or more antigen parameter population immunophenotyping 125 for each event population 100. Populations are classified according to a set of criteria that account for all antigen parameters 30 in the datasets 10. The classification criteria are based on literature, standard practice in the field, and, in some instances, statistical data. These criteria must be customized based on the set of parameters in the assay. The criteria include MFIs, expression status (i.e., positive, negative, or equivocal), and, for B-cell populations, light chain expression 124. The cell type of each population and whether it is normal or abnormal is determined. For example, according to one such set of criteria if a population is positive for CD45, CD19, CD20, CD5, and kappa, and is negative for all other antigen parameters, the population may be classified as a “B-cell neoplasm.” In the previous example, determination of whether a population is positive or not for the foregoing identified antigen parameters 30, may be based on a ratio cutoff such as at least 20% of events exceeding a classification antigen parameter positivity cutoff value. The positivity cutoff value may be determined based on an isotype control or internal negative control population (e.g., fluorescence values below which 95% of events of the negative control population fall). Other exemplary population classifications 121 may include CD5+B-cell neoplasm, CD4 positive T cells, NK cells, B-cell lymphoma, acute myeloid leukemia, and many others that are well documented and known in the medical field. Population classifications 121 may be dataset population classification 121 for populations within a datasets 10, preliminary population classifications 121 across multiple datasets, and final population classifications 121 for all populations across combined dataset 11 for a full patient sample.

Embodiments of the present invention may also be configured to Detect and merge duplicate event populations. Detection and merging of duplicate populations may be performed by combining the events of every two evaluated event populations into a single assessed population and performing the clustering analysis 500 described above on the events of the assessed population. If no distinct subpopulation is detected within the assessed population, the evaluated event populations are confirmed to be duplicate populations and their events 20 are merged into one event population. If a distinct population is detected, the evaluated populations are confirmed to be distinct and are not merged. Optionally, populations with antigen MFI differences exceeding pre-defined cutoff values may be assumed to represent distinct populations and merging attempts may be bypassed.

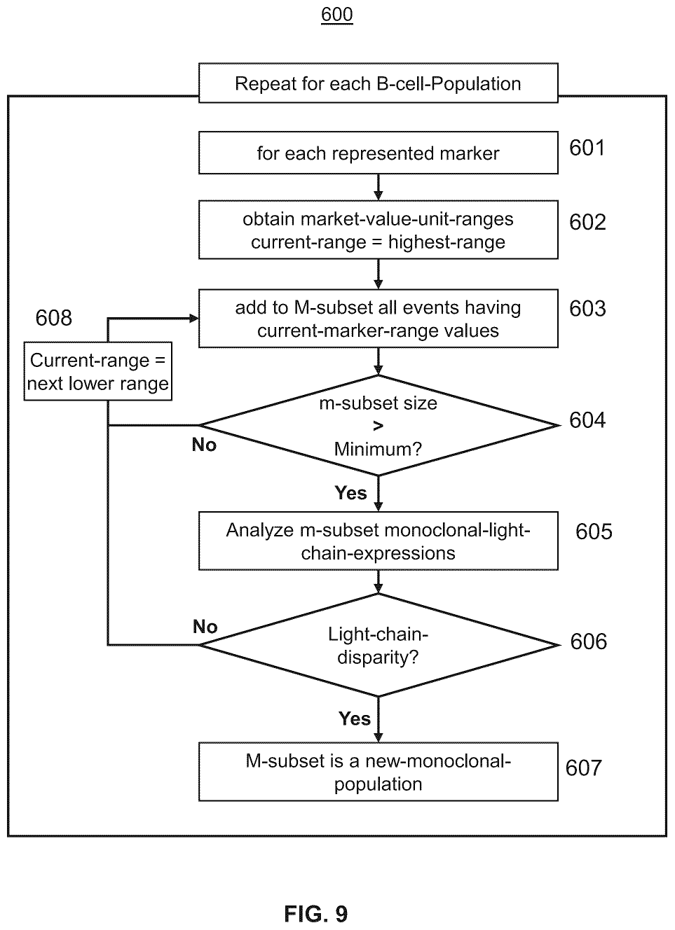

Embodiments of the present invention, as illustrated in FIG. 9, may be configured to perform a monoclonal subset analysis (“M-subset analysis”) 600 to evaluate each B-cell population in a dataset 10 for the presence of monoclonal subpopulations. Monoclonal subpopulations may represent distinct subpopulations that may not have been detected during a previous clustering analysis 500. Each B-cell population is divided into monoclonal parameter positivity subsets based on the kappa and lambda levels of expression and analyzed for differences in light chain expressions 124 using the following steps:

M-subset analysis 600 may be repeated for one or more parameters represented in a B-cell population as indicated in step 601. In step 602 computing system 2 obtains (e.g., accesses, calculates, determines, retrieves, receives) a number (e.g., 20, M, N, X-count, Y-count) of parameter value unit ranges (corresponding to, e.g., x-unit range or y-unit range) and ranging from lowest (e.g., X-min, Y-min) to highest parameter value unit range (e.g. X-max, y-max). In step 602, computing system 2 may assign the highest parameter value range as a current range.

In step 603, M-subset analysis 600 may identify in the B-cell population events 20 with parameter values 31 for a represented parameter (i.e., represented in the population) within the current range (initially, e.g., the highest value range, the 20^{th} highest unit range, etc.) and add those events to a monoclonal subset. In step 604 computing system 2 may determine if the M-subset size (e.g., number of events) exceeds a minimum M-subset size. If there are no M-subset events, or not enough events, indicated as “No” in step 604, the process returns to step 603, but as indicated in step 608, using as a current range the next highest parameter value unit range. By way of an example, if using a 20 by 20 coordinate system 60, the next highest parameter value unit range would be the 19th, 18th, and so on. M-subset analysis 600 then repeats step 604 to add to the M-subset events with parameter values 31 within the current range (e.g., 19th, 18th, etc.) and determine the M-subset size. This process of adding to the monoclonal subset events with parameter values 31 within each subsequently lower current range continues until the monoclonal subset comprises a minimum M-subset size as indicated with “Yes” in step 604. The minimum M-subset size may be adjusted for a desired sensitivity of analysis 600.

In step 606, the monoclonal subset is analyzed to identify a monoclonal light chain expression indicator representative of a light chain expression disparity. For example, such a monoclonal light chain expression indicator may indicate that the analyzed monoclonal subset is monotypic, while the parent B-cell population is polytypic, that the monoclonal subset may be kappa monotypic while the B-cell population is lambda monotypic, and other light chain inconsistencies that are well known in the field. If a monoclonal light chain expression or light chain inconsistency is identified, the events in the monoclonal subset are separated as a distinct monoclonal population. The monoclonal analysis of the B-cell Populations may be performed on the next B-cell population of the combined dataset 11.

If a monoclonal light chain expression indicator is not identified (e.g., no light chain expression disparity), as indicated in step 606 with “No,” then the process returns to step 603, but as indicated in step 608, using the next lower parameter value unit range as the current range. In step 603 events parameter values within the current range are added to the monoclonal subset, and the subset is re analyzed in step 605 for a monoclonal light chain expression to determine if the monoclonal subset is a distinct population or not. If no light chain expression disparities (or abnormalities) are found, process 600 repeats until all events of the B-cell population have been added to the monoclonal subset. The monoclonal analysis 600 may be repeated in the same manner but starting with the events with parameter values 31 in the lowest parameter value unit range as a current range, and increasing the current range to include successively higher parameter value 31 unit ranges.

Computing system 2 may be configured to determine a population size ratio 122 for each event population, the population size ratio 122 representative of the percentage of the population size 105 from the number of events in a dataset 10, preferably after exclusion of debris, doublets, and other undesirable events. Each population size ratio 122 may also be determined as a percentage from all events in a combined dataset 11 of several tube samples.

Each of the clustering algorithm 500; the determination of an immunophenotype 125 for each population 100; the detection and merging of duplicate populations 100; and the monoclonal subset analysis 600, may be performed on each of all available datasets 10, each dataset representing a sample tube from a patient sample, and all sample tube datasets 10 together may represent a combined dataset 11 from a full patient sample. When analyses on each sample tube dataset 10 are complete, the computing system 2 may be configured to analyze events 20 in populations 100 of a combined dataset 11.

For example, a full population immunophenotype 125 may be determined for each event population 100 in a combined dataset 11, based on one or more of i) an antigen MFI for each antigen parameter 30 represented in each event population, ii) an antigen parameter expression, iii) an antigen parameter intensity, and iv) a light chain expression.

To determine a full population immunophenotype 125 of event populations 100 across a combined dataset 11 may require an analysis of parameters tested in different sample tubes and received by computing system 2 as part of events 20 in separate datasets 10. In one embodiment, to assess the status of parameters from separate datasets 10 (e.g., distinct files) a bivariate data of CD45 and side scatter may be analyzed (e.g., by representing the data in a coordinate system 60) to find a region with maximum proportion of the population being evaluated. The bivariate data in a data structure representing a coordinate system is divided into a series of units, analogous to clustering analysis 500, and the event density of the population in each unit is calculated. The proportion of the population of interest out of all events can then be calculated.

A range of forward scatter parameter values (e.g., forward scatter fluorescence) that encompasses a large proportion of events (more than, e.g., 40%, 50%, 75%, etc., events) of a population in interest may be determined. The forward scatter parameter value range may be based on a statistical calculation (e.g., excluding the top and bottom 1% of events or events outside of two standard deviations, etc.). If there are any other parameters 30 in common between datasets 10, then fluorescence ranges of parameter values that encompass a large portion (e.g., more than half, ⅔, 45%, etc.) of events 20 in the population are also determined for these in common parameters 30. Using such fluorescence ranges for the parameter values may enrich the sample of events 20 to be evaluated in other datasets 10 with the population of interest.

In one embodiment, the ranges of parameter values 31 for parameters CD45, side scatter, forward scatter, and any other parameters are then applied to other datasets 10. The proportion (or number, quantity, count) of events 20 positive for a particular parameter may be calculated. If the proportion of events is less than a cutoff to determine positivity when assuming that all of the positive signal is on the population of interest, then it is assumed to be negative. If the proportion of events with this parameter exceeds the positivity cutoff when assuming that all of the negative signal is on this population, it is assumed to be positive. If neither of these two criteria are met, the population of interest is labeled equivocal.

Embodiments of the invention may perform a final immunophenotyping process 700 to determine a final population immunophenotype 125 for each population in a combined dataset. The initial steps of final immunophenotyping 700 are analogous to the steps for determining population immunophenotypes 125 described above. For example, the proportion of events positive for the parameter 30 being assessed is calculated within the range of parameters CD45, side scatter, forward scatter, and, if relevant, other parameters to enrich the sample with the population being evaluated. During final immunophenotyping 700, expression statuses of parameters across datasets are assessed accounting for the population immunophenotypes 125 of all populations in a combined dataset, providing a higher degree of accuracy. For final immunophenotyping 700, computing system 2 may be configured to only assess parameters with equivocal parameter expression status to determine whether a positive or negative parameter expression status can be determined based on all data available across the combined dataset.

Each unique population having a positive or negative parameter expression status for a parameter 30 being assessed is evaluated. Known positive populations are subtracted from the proportion of positive events, and known negative populations are subtracted from the proportion of negative events. The remaining positive and negative events, which still could potentially belong to the population being evaluated, are then analyzed.

If the proportion of the parameter 30 is less than the cutoff (or limit) to determine positivity when assuming that all of the positive signal is on the population of interest, then it is assumed to be negative. If the proportion of the parameter 30 exceeds the positivity cutoff when assuming that all of the negative signal is on this population, it is assumed to be positive. If neither of these two criteria are met, it the population of interest is labeled equivocal.

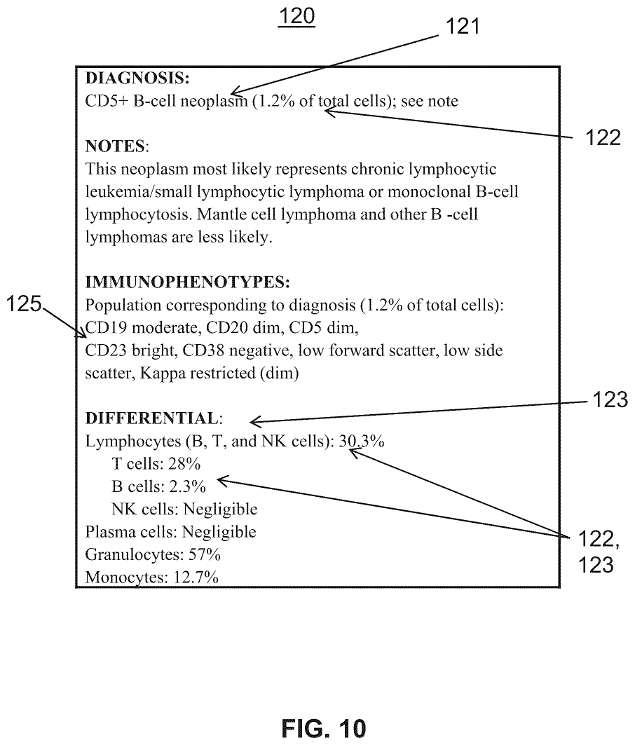

FIG. 10 illustrates several population immunophenotypes (e.g., CD19 moderate, CD20 dim, CD5 dim, CD23 bright, CD38 negative, low forward scatter, low side scatter, Kappa restricted (dim)). Population immunophenotypes may be based on populations in a dataset, on populations in multiple datasets 10, or on all populations in a combined dataset 11. For diagnostic purposes, preferably, population immunophenotypes based on all populations in a combined dataset are used.

A preliminary classification of all event populations in the combined dataset 11 may be determined based on a set of rules used to identify a sample tube dataset from which a population originates (or was reported to originate from) to prevent double reporting of event populations. Same abnormal population can often be detected in multiple datasets, but for proper diagnosis, each abnormal population must be reported only once. The set of preliminary classification rules may be designed to optimize the characterization of populations by reporting from a dataset each population including the most diagnostically relevant set of parameters for that particular population (e.g., report T cell populations from a tube that includes multiple T cell antibodies). Datasets may include parameters that optimally separate a particular population from other populations during clustering analysis. For example, if a population is found to be positive for CD19, it is likely a B-cell population. A preliminary classification rule can be used such that CD19 positive populations are only reported from the tube that includes other B-cell parameters such as CD20, kappa, and lambda. If the tube contains parameters that are typically positive on this type of population, this allows it to be separated from other populations during clustering analysis.

Another set of criteria may be used to determine whether each population may be a unique population among all populations detected in all datasets 10. This information is later used during final immunophenotyping. This determination is necessary, as each population must only be accounted for once when assessing percentages of events expressing each parameter, as further illustrated with respect to final immunophenotyping 700. These criteria must be customized based on the set of parameters in the assay. The criteria can include MFIs and expression status (i.e., positive, negative, or equivocal). For example, if a population is positive for CD19, it can be assigned as a unique population in the first tube that includes CD19. If a population detected in any other tube is found to be positive for CD19, it is not considered unique.

Following a final immunophenotyping 700 of all event populations across the combined dataset, a final population classification 121 for all populations may be determined using the same methodology as for preliminary classification described in the preceding paragraphs, but when performing a final population classification 121 the computing system 2 is configured to account for the final immunophenotype 125 of each population.

Computing system 2 may be configured to detect duplicate populations in a combined dataset by creating combinations of every two populations from different datasets 10 and comparing the parameter expression status of each parameter common in both populations. If any of the parameter expression statuses (e.g., positive, negative, equivocal) differ for the same parameter between the populations, then the populations are not considered potential duplicates. Forward scatter, side scatter, and any antibody parameters in common between the datasets may be compared using population parameter MFI rather than expression positivity status. Predetermined duplicate ranges and/or thresholds are used to determine whether the population parameter MFIs may indicate potential duplicate populations. Such duplicate ranges and/or thresholds may be empirical and determined by comparing MFIs of the same population detected in multiple tubes and calculating statistical cutoffs (e.g., greater than two standard deviations from the mean expected MFI difference).

Following a final immunophenotyping 700 of all event populations across a combined dataset, a final duplicate population detection may be performed, consistent with the preceding description, but accounting for the final immunophenotype 125 of each population.

Computing system 2 and user interface 4 are configured to generate a report 120 (e.g., as a text file, image, etc.) comprising diagnoses, notes, immunophenotypes, and differential counts 123, preferably for the combined dataset 11 representative of a patient sample. Report 120 may be based on the one or more population classifications 121, population immunophenotyping 125, light chain expressions 124, and population size ratios 122. Diagnoses and notes may be generated to account for all detected classification abnormalities, for example neoplasms (e.g., lymphoma, acute leukemia), numerical abnormalities (e.g., lymphocytosis, monocytosis), and various others. Diagnoses and notes are based on literature (e.g., WHO classifications) and standard practice in hematopathology. For example, embodiments of computing system 2 may be configured to access digital diagnostic data (e.g., obtained from World Health Organization (WHO) classifications, hematology references, and other sources) to generate report diagnoses and notes consistent with the populations' analyses, such as population classifications 121, population immunophenotypes 125, light chain expressions 124, population size ratios 122, and others. Embodiments may be configured to store the digital diagnostic data on datastore 3, access it from a portable storage device, or computing system 2 may access the digital diagnostic data over a network.

Information about possible diagnoses and notes and corresponding population classifications 121 may be stored on the datastore or may be available over a network connection. The user interface is configured to access the diagnoses and notes information and extract the appropriate diagnose and/or note based on the population classification, and diagnostic criteria that if available may be used to access and provide optimal diagnoses and notes (e.g., explanatory note) for each abnormal final population classification. Such diagnostic criteria may include the specimen type (e.g., bone marrow, blood, patent's age, the final population immunophenotype, the population percentage from the entire sample dataset, and others). The diagnostic criteria may also account for other populations detected in the sample, which can sometimes affect the diagnosis or the wording of the diagnosis or note. Notes may also be generated to indicate which populations may represent duplicates based on the results of duplicate detection.

Another report section may describe the final population immunophenotype 125 of each abnormal population by listing each parameter and whether the parameter's expression status, and intensity (i.e., dim, moderate, or bright), as illustrated in FIG. 10 under the heading “IMMUNOPHENOTYPES.”

In some embodiments, report 120 may comprise a recommendation for an additional analysis by configuring computing system 2 to detect certain diagnoses, classifications, or immunophenotypes and if detected, to generate an appropriate recommendation. For example, if report 120 includes a diagnosis of B-cell lymphoma computing system 2 may be configured to include a recommendation in report 120 that an additional sample tube of B-cell markers should be run through flow cytometer 6 and the sample tube dataset 10 analyzed through a system according to this invention. The recommendation may also suggest that a pathologist manually review some or all of the diagnostic data.

As illustrated in FIG. 10, the report 120 may also comprise a differential count 123 for a cell type (e.g., Lymphocytes, T Cells, B Cells, NK, etc.), generated based on the percentages of populations having the cell type classification detected in the combined dataset 11.

While the invention has been described with reference to exemplary embodiments, it will be understood by those skilled in the art that various changes, omissions, and/or additions may be made and equivalents may be substituted for elements thereof without departing from the spirit and scope of the invention. In addition, many modifications may be made to adapt a particular situation or material to the teachings of the invention without departing from the scope thereof. Therefore, it is intended that the invention not be limited to the particular embodiments disclosed as the best mode contemplated for carrying out this invention, but that the invention will include all embodiments falling within the scope of the appended claims. Moreover, unless specifically stated, any use of the terms first, second, etc. do not denote any order or importance, but rather the terms first, second, etc. are used to distinguish one element from another.

I claim:

1. An apparatus for analyzing flow cytometry data for a fluid sample, the apparatus comprising:
a datastore that stores one or more cytometry datasets, each cytometry dataset comprising a series of events about the fluid sample;
wherein each event comprises a plurality of parameter values;
wherein each of the plurality of the parameter values is associated with one parameter from a plurality of parameters;
wherein the plurality of parameters comprises scatter parameters and antigen parameters;
a computing system coupled to the datastore, wherein the computing system operates upon the one or more cytometry datasets;
one or more marker pairs, each marker pair comprising a first parameter and a second parameter;
wherein the first parameter and the second parameter are selected from the plurality of parameters;
a user interface coupled with the computing system;
a data structure representing a bivariate coordinate system having an x-axis, a y-axis, and an area;
wherein the area comprises units, each of the units having an x-unit range and a y-unit range;
wherein the units comprise cluster units and empty units; and,
one or more event populations selected from a group consisting of an initial population and one or more subpopulations;
wherein each of the one or more event populations comprise one or more clustered events from the series of events; and,
wherein for each marker pair the computing system is configured to:
(1) for each of the one or more event populations, assign each of the clustered events to one of the cluster units based on the parameter value for the first parameter of each clustered event, the parameter value for the second parameter of each clustered event, the x-unit range of each cluster unit, and the y-unit range of each cluster unit;
wherein each cluster unit comprises at least one clustered event and a cluster event density representative of the number of clustered events in the cluster unit;
(2) determine the event population for each of the cluster units from the one or more event populations based on one or more of i) the cluster event density, ii) a closest cluster population, iii) a distance to closest population, and iv) one or more intervening density differences;
(3) determine an immunophenotype for each event population based on one or more of i) an antigen median fluorescence intensity for each antigen parameter represented in the event population, ii) an antigen parameter expression, iii) an antigen parameter intensity, and iv) a light chain expression;
(4) determine one or more population classifications based on the immunophenotype for each event population; and,
(5) generate a diagnostic report based on the one or more population classifications.

2. The apparatus of claim 1, wherein each event represents a cell in the fluid sample;
wherein each of the one or more event populations comprises a population size greater than a minimum analyzable population size;
wherein the bivariate coordinate system comprises X-count units along the x-axis and Y-count units along the y-axis;
wherein each clustered event is assigned to the one cluster unit if the parameter values of the clustered event are within the x-unit range and y-unit range of the cluster unit; and,
wherein the computing system sorts the plurality of the cluster units according to the cluster event density of each cluster unit.

3. The apparatus of claim 2, wherein each antigen parameter is associated with a parameter cutoff value;
wherein each of the one or more event populations comprises a quantity of marker pair positive events;
wherein the quantity of marker pair positive events is greater than a minimum event count;
wherein each of the marker pair positive events comprises the parameter value from the plurality of parameter values greater than the parameter cutoff value; and,
wherein the parameter value is one of i) the first marker pair parameter value and ii) the second marker pair parameter value.

4. The apparatus of claim 3, wherein the distance to closest population represents the distance that is shortest between the cluster unit and a closest assigned cluster unit from the plurality of cluster units;
wherein the closest assigned cluster unit comprises the closest cluster population;
wherein the closest cluster population is selected from the one or more event populations;
wherein the event population is the same as the closest cluster population if the distance to closest population is less than a same population distance limit;
wherein the event population is a new subpopulation if the distance to closest population is greater than a new population distance limit; and,
wherein the computing system is configured to determine the event population based on the intervening density differences between the cluster unit and one or more intervening cluster units if the distance to closest population is between the new population distance limit and the same population distance limit.

5. The apparatus of claim 4 further comprising the one or more intervening cluster units;
wherein the one or more intervening density differences are density differences between the cluster unit and each of one or more evaluated intervening cluster units; and,
wherein the one or more evaluated intervening cluster units are selected from among the one or more intervening cluster units.

6. The apparatus of claim 5, wherein each of the intervening density differences is associated with one of the evaluated intervening cluster units and with an associated intervening density difference cutoff;
wherein the event population is the closest cluster population if each of the intervening density differences is less than or equal to its associated intervening density difference cutoff; and,
wherein the event population is the new subpopulation if at least one of the intervening density differences is greater than its associated intervening density difference cutoff.

7. The apparatus of claim 6, wherein the x-unit range of each intervening cluster unit is between the x-unit range of the cluster unit and the x-unit range of the closest assigned cluster unit;
wherein the y-unit range of each intervening cluster unit is between the y-unit range of the cluster unit and the y-unit range of the closest assigned cluster unit; and,
wherein the one or more evaluated intervening cluster units are selected from the group consisting of i) all intervening cluster units, ii) intervening cluster units that are crossed by a straight line connecting the cluster unit and the closest assigned cluster unit, and iii) one or more randomly selected evaluated intervening cluster units from among the one or more intervening cluster units.

8. The apparatus of claim 7, wherein the area is a square and comprises an equal number of units along the x-axis, and along the y-axis.

9. The apparatus of claim 8, wherein the area comprises 400 units.

10. The apparatus of claim 1, wherein each of a plurality of antigen expressions is determined based on an antigen MFI value for each of the antigen parameters in the event population;
wherein each of the plurality of antigen expressions is selected from a positive antigen expression, a negative antigen expression, and an equivocal antigen expression;
wherein each of a plurality of antigen parameter intensities is determined based on the antigen MFI values for an antigen parameter having the positive antigen expression; and,
wherein each of the plurality of antigen parameter intensities is selected from a dim intensity, a moderate intensity, and a bright intensity.

11. The apparatus of claim 10, wherein the event populations further comprise a B-cell population;
wherein the B-cell population comprises positive antigen expressions for B-cell parameters;
wherein the B-cell parameters are selected from the group consisting of CD19, CD20, CD22, CD79a, CD79b, and combinations thereof; and,
wherein the computing system is configured to determine one or more light chain expression ratios for each of the B-cell populations based on a lambda parameter value and a kappa parameter value.

12. The apparatus of claim 11, wherein each of the one or more event populations comprises a population size ratio; and,
wherein each population size ratio is representative of a ratio of each event population size to the number of events in the series of events.

13. The apparatus of claim 12, wherein the diagnostic report is generated based on one or more of i) population classifications, ii) population immunophenotyping, iii) light chain expression ratios, and iv) population size ratios.

14. The apparatus of claim 13, wherein the B-cell parameters are further selected from the group consisting of CD24, CD27, PARS, OCT2, BOB1, immunoglobulin, and combinations thereof.
